(19) United States
(12) Patent Application Publication
(10) Pub. No.: US 2019/0133086 A1
(43) Pub. Date: May 9, 2019

(54) HORSE MONITOR SYSTEM AND METHOD

(71) Applicant: PELLESUS LTD., Industrial park Hevel Modiin (IL)

(72) Inventors: Yoav Katz, Ginaton (IL); Moti Itzkovich, Petach-Tikva (IL); Nathan Sela, Ness Ziona (IL)

(21) Appl. No.: 16/164,884

(22) Filed: Oct. 19, 2018

Related U.S. Application Data
(60) Provisional application No. 62/583,023, filed on Nov. 8, 2017.

(51) Int. Cl. A01K 29/00; A01L 15/00; A61B 5/11; A61B 5/00; A61B 5/01

(52) U.S. Cl. CPC A01K 29/005; A01L 15/00; A61B 5/11; A61B 5/7267; A61B 2503/40; A61B 5/1123; A61B 2562/0219; A61B 2560/0242; A61B 5/01

(57) ABSTRACT

A method for evaluating a motion related parameter of a horse, the method may include generating, by multiple sensing units attached to multiple legs of the horse, sensor information; transmitting the sensor information; receiving, by a remote computer, the sensor information; and evaluating, by the remote computer, the motion related parameter of the horse by applying, on the sensor information, a machine learning process trained on a training set that comprises (a) sensor information that represent a desired motion related parameter and (b) sensor information that represents deviations from the desired motion related parameter.

### CROSS REFERENCE

This application claims priority from U.S. provisional patent Ser. No. 62/583,023, filing date Nov. 8 2017.

### BACKGROUND

Horses are delicate and costly animals that tend to get injured and/or get sick.

There is a growing need to provide effective systems and methods for monitoring horses.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 illustrates an example of a horse, a system and its environment;

FIG. 2 illustrates an example of a horse, and some part of the system;

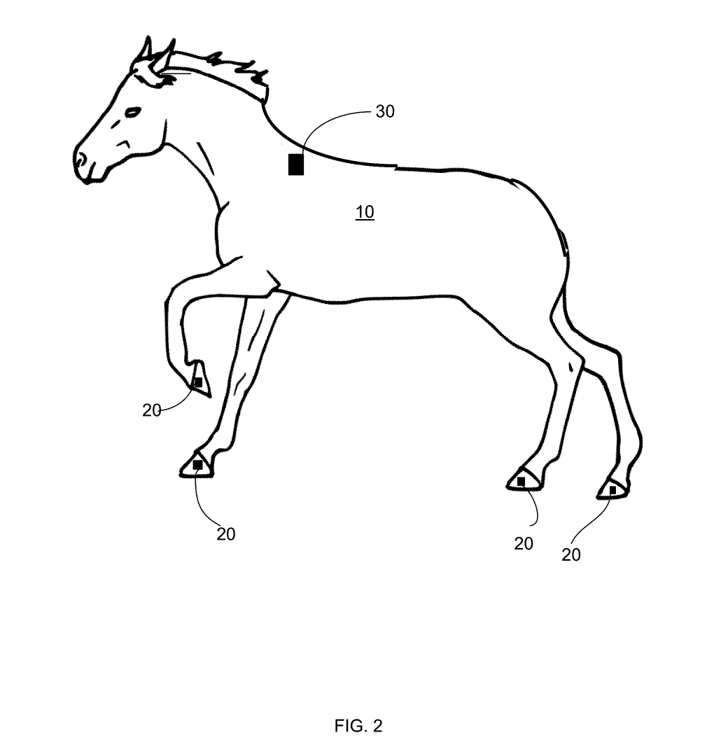

FIG. 3 illustrates an example of a method;

FIG. 4 illustrates an example of a method; and

FIG. 5 illustrates an example of a method.

### DETAILED DESCRIPTION OF THE DRAWINGS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference to a method may be applied mutatis mutandis to a system capable of executing the method and/or to computer program product that stores instructions for executing the method.

There may be provided a method for monitoring horses or any other quadruped. The method may use big data analysis and/or machine learning algorithms that may collect and process a vast amount of information (including motion related information) that is obtained over long period of time regarding many horses. The method may build and/or update any number of profiles related to:
a. One or more specific horses.
b. One or more horses that belong to a specific brand.
c. One or more horses that belong to a specific brand and are of specific gender.
d. One or more horses that belong to a specific brand and are of a specific age.
e. A general horse profile.

The method may generate any profile for any group of horses that fulfill a certain combination of parameters.

Building profiles may be based upon associating sensor information to a certain horse, or to horses of certain parameters (age, gender, brand) and then processing the data associated with this certain horse and/or to horses of that certain parameters.

The profile may be generated based on information about one or more horses. For at least one horse the information may include motion related information and additional information.

The motion related information may be sensor readings and/or processed sensor readings. The sensor readings may be obtained by one or more sensors that may include any nine degrees of freedom (9-DOF) sensor that may combine a 3-axis accelerometer with a 3-axis gyroscope with a 3-axis magnetometer.

The 9-DOF sensor may be replaced by another degree of freedom sensors.

The readings of the sensors may be processed in various manners—for example:
a. Reconstruction of the horse gait from a sequence of coordinates samples of the four hooves. The reliable horse movements is based on inverse kinetics.
b. Determining the orientation of the different legs of the horse—and comparing the orientation readings.
c. Taking into account the cyclic nature of the movement of the horse to filter noises, improve the signal to noise ratio, and/or on order to zero various drifts.

The readings may be processed for restraining the error accumulation of the sensors. For example, position error is proportional to time squared. Without an error-resetting algorithm, this error can exceed a meter in ten seconds.

A deceleration-acceleration algorithm will be employed if the sensor is mounted on a hoof. During normal walking trotting, Galloping, four bits, etc. cycles, a limb periodically returns to a stationary state and remains on the ground for a brief period of time (depend on the horse speed); this interval is referred to as the zero velocity interval. When a stationary state is detected, the velocity error can be employed as an observation to estimate and can correct the sensor bias errors.

At least one sensor may be attached to each leg of the horse. Other sensor may be attached to additional or other locations of the horse. See, for example US patent application 2007/0130893.

The additional information may include at least one out of:
a. Feedback received from any person (owners and /or caretakers such as veterinarians and/or jockeys and/or trainers) about the health and/or performance of the horse.
b. Feedback from other sensors such as cameras, GPS or other location sensors, ambient condition sensors, and the like.
c. Information about the terrain. The terrain may affect the movement of the horse. For example, climbing a steep and rocky mountain differs than running along a flat and dry surface. There may be provided a mapping between the location of the horse and the terrain.

The motion related information regarding a certain horse may be processed by applying at least one of the following methods:
a. Comparing between readings of different motion sensors coupled to the same horse. For example, a consistent difference between a movement of one leg to another may represent a problem.
b. Change in the horse's movement pattern like dramatic reduction of the number of steps per unit time.

The profiles which are generated based on on a vast number of parameters and, especially when provided feedback from other sources, may indicate about injuries and/or other health problems—even before said problems and/or injuries are recognized by the horse caretakers.

Finding such injuries and/or health problems may be implemented by (i) searching for a feedback that was given at a certain point in time and is indicative of an injury and/or health problem that was recognized by a horse caretaker, (ii) finding the motion related information associated with that that certain point in time, (ii) searching for motion related information that was obtained before that certain point of time that may be used as a signature of the injuries and/or health problems—even before they are recognizable by the horse caretakers.

The motion related information is very valuable—to the horse caretakers as well as to other parties—including competitors, gambles, and the like.

The motion related information is conveyed from a sensing unit in a secure manner and may be automatically deleted from the sensing unit after transmission—or after a certain period of time following the transmission—in order to reduce the chances of downloading the motion related information from the sensing unit.

The sensing unit may include:
a. A sensor
b. A signal processor for processing sensor readings and generate the coordinates related information
c. A controller for energy management, for synchronizing between sensors attached to the same horse, for access control—including encryption of the motion related energy and deletion of the motion related energy, and for communicating with another communication device that may further relay or transmit the encrypted motion related information to another network, another computer, to the cloud, over the Internet and the like.

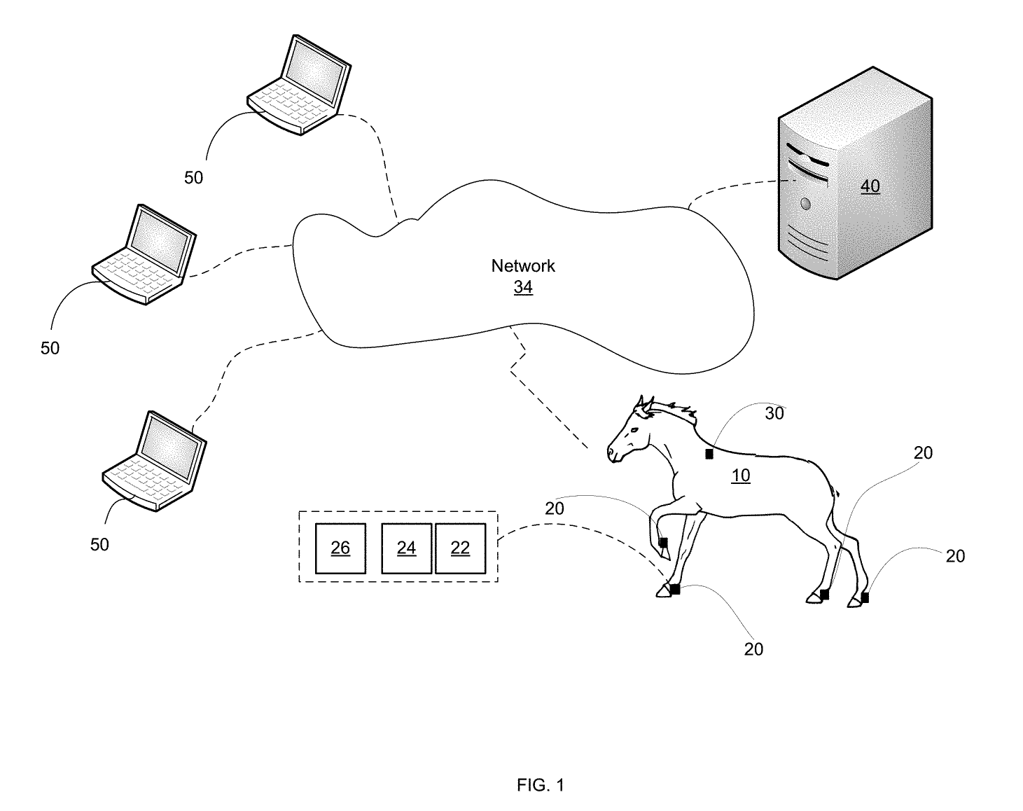

FIG. 1 illustrates a horse 10 equipped with four sensing units 20, a communication module 30, network 34, a cloud computerized system 40, additional feedback sources 50 that may include any of the mentioned above feedback sources. Each sensing unit may include motion sensor 22, signal processor 24 and controller 26.

The sensing units 20 may be attached to the hooves or near the hooves of the horse.

There may be provided a method for monitoring a horse, the method may include sensing motion of the horse by one of more sensors, processing the sensors reading to provide motion related information, encrypting the motion related information, transmitting, in a secure manner, the motion related information, deleting the motion related information from the sensing unit.

There may be provided a method for monitoring horse.

The method may include:
a. Obtaining motion related information that is related to a movement of the horse.
b. Obtaining additional information such as any of the feedback listed above.

Comparing the movement of the horse to a profile that is relevant to the horse in order to find deviations that may indicate of an injury, a health problem or any other event of interest.

Generating an alert or any type of information or indication about the outcome of the monitoring.

The outcome of the monitoring may be sent to any of the horse caretakers, may be distributed between veterinarians, and the like. The method may select a suitable professional to solve any problem—based, for example, on a distance between the professional and the horse, on the availability of the professional, on the expertise of the professional, and the like.

The suggested methods, systems and computer program products may be used in various fields—including, for example, Biomechanics based on motion capture aiming to the horse performances optimization in various horse sports.

The reading of the sensors may be fed to as an input to a 3D computer graphics animation based on the 9D sensors motion capture.

Hooves trimming

The methods, systems and computer program product may be used for providing hooves trimming recommendations based on motion capture and ML analysis-Hooves trimming is a knowhow professionality.

Trimming recommendations based on optimizing the hoof's angle position and orientation on the ground will be done by analyzing the motion captured data.

Gait analysis

Kinematic information on the movement of each hoof. Two of these sensors are accelerometers and gyroscope. The accelerometer measures the 3-axial acceleration relative to the sensor frame of reference. By “frame of reference” we mean the local 3 dimensional X, Y, Z frame of the sensor body which changes when the sensor is changing its orientation. The gyroscope, also, measures the 3-axial rate of rotation also relative to the sensors frame of reference. By combining of the readings of both these sensors, as set forth in the next bullets, it is possible to calculate the full 6DOF pertaining to the location and orientation of the sensor in space. The fusion procedure comprises of the following stages:
a. Integration of the gyroscope readings to obtain the absolute orientation of the sensor in space (provided that appropriate initial conditions are used, which means that during the static phase before moving the movement one can calculate the initial orientation of the sensor using the fact that in static condition, the accelerometer measures only the gravitation g)
b. Conversion of the measured 3 axial acceleration from the sensor frame of reference to the earth frame of reference by multiplying it with the orientation conversion matrix
c. Subtracting earth's gravity −g
d. The resulting is the “net” linear acceleration which represents the 3-dimensional acceleration of the sensor relative to the earth's frame of reference
e. As is known, acceleration is the second derivative of the location thus when the acceleration is known one can get the location as a function of time (i.e. trajectory) by double integration of the acceleration in time to yield (using appropriate initial and boundary conditions created based on sensors data during stance periods) the 3-dimensional trajectory of the sensor which actually represents the trajectory of the hoof in space.

All in all, this procedure results in the 3D trajectory of the hoof as well as the 3D orientation of the hoof during swing phase.

The 3D orientation of the sensor is representative of the orientation of the horse hoof during swing phase. The latter can provide very important biomechanical information on the normal or abnormal behavior of the horse gait. For example, a lame horse is usually limited in the degrees of freedom of its hoof during swing, and this is reflected in the limited variability of the hoof orientation during that stage. Thus, the so called “foot fall” which may indicate whether a horse hoof has been trimmed correctly or not. For example, a rapid change in the horse “footfall” which is detected short time after trimming of the horse hoof (relative to its footfall prior to trimming) is a clear indication that the trimming need to be rectified.

One of the aspects of the present invention is therefore to use the three-dimensional angular information of the horse hoof during swing phase to detect abnormal “foot fall” orientations, which may be indicative of the need to trim the horse hooves or whether a previous trimming need to be redone and how to be redone. It may be also indicative to a medical issue. Such an alert can be generated via the central processing unit and transmitted to the horse owner and caretakers, particularly the hoof trimmer.

Another fundamental aspect of the present invention is the combination of data from all four limbs of the horse, as well as other supportive data, such as but not limited to, temperature, pulse, moisture, pressure etc., in order to generate a more sensitive and specific medical assessment. For example, a horse that suffers from lameness in one of its limbs may show changes in the gait of other limbs too, such healthy limbs are actually compensating for the lame limb. The changes are defined relative to the healthy baseline of the specific horse, such data exists in the system data base because each horse is monitored 24/7. This can be manifested in a shorter stance period of the lame limb whereas the healthy limb of the opposite side shows typically a longer than normal stance period. Thus, diagnosis of lameness is better diagnosed by combining the sensors information from all four limbs, looking for anomalies in a comparative manner. In addition, colic conditions are characterized by a restless horse which kicks the ground using one of its front limbs. Thus, the typical signs of colic can be verified in a comparative way to other limbs (sensors) which are stationary during the time that the first limb is kicking. This emphasizes the importance of analyzing the data from all four sensors in a synergetic way, which provides added information, which does not exist if each limb is analyzed separately.

Also, a lame limb often is characterized by prolonged elevated limb temperatures, which is indicative of a local inflammation, so continuous measurement of all four limbs temperatures, which should be measured directly on the hoof which is where the sensors are fixed (as well as core body temperature and environmental temperature) can support diagnosis of lameness by showing temperature differences between the lame limb and other limbs. Such temperature difference can be measured simpler than the absolute temperatures.

Lameness can be analyzed in two different by complementary types of analysis, which can be implemented in a synergetic manner:

Individual analysis in a comparative manner to its own “healthy baseline” inertial signature

Big data analytics which will be accumulated from a large group of “labeled by experts” horses and processed by appropriately trained machine learning algorithms

It is yet another aspect of the present invention that the gait rhythm of the horse has to be measured and identified. This is an important feature of lameness detection which, mainly in mild lameness, depends on the gait. This means that mild lameness (lameness grade 1 or 2) may not be visible during walk and begins to be visible in trot or faster gaits.

In the present invention the gait can be identified by a complementary and synergetic types of analysis:

Detect the timing of swing/stance phases of each limb and perform phase analysis between all four limbs, taking advantage that each gait is characterized by a particular intra-limb rhythm. This may be achieved as follows: the stance is characterized by a static limb on the ground thus the acceleration which is measured by the accelerometer during stance is the gravity g. When the limb is moving during swing the acceleration deviates from g. Thus, the segmentation to stance phases and swing phases of each limb can be done on the basis of accelerometer readings. Now, since all four sensors on four limbs are synchronized, the system can actually track and determine the stance and swing phases of each limb on the same time line. This allows the determination of the gait rhythm at each gait since each gait has different attributes for example in walk gait the order of swinging limbs is 4 beat—left hind, left front, right hind, right front and so on. The trot is two beat, moving diagonal pairs of limbs simultaneously etc.

Collect data from a plurality of horses in a variety of gait rhythms and train a machine learning algorithm to identify the gait

The identified gait is later fed into a machine learning algorithm which is trained to detect lameness and classify the degree of severity of the latter

It is another aspect of the present invention that the sensors are attached to the horse such as to provide a continuous 24/7 monitoring. This dictates that the sensors will be attached onto a location in the horse body which will not cause the animal any discomfort or stress during a prolonged period of time, nor will it be captured or trapped in any mechanical object existing in the everyday environment of the horse. To this end, in this invention it is suggested that the at least four sensors will be attached to the horse hooves using a biologically compatible glue which on one hand allows a strong and stable attachment to the hoof and on the other hand allowing release (by any means which are acceptable such as chemical and/or mechanical etc.) and regluing of the sensor when replacing battery or moving it to its original location following growth of the hoof and trimming. This particular location of the sensor together with a small form factor will protect the limb from being trapped or captured. In addition, the sensors must be protected against horse bites and leg-onto-leg rubbing or brushing against, which a bored animal or a restless one is expected to do.

It is also another aspect of the present invention that the machine learning algorithms will be trained to detect a variety of horse conditions, each of which may be related to different point of reference and each may have a different degree of urgency. For example, detection of a life-threatening condition such as colic will require the system to generate an immediate alert, via cellular communication means such as GSM, LTE etc. in cases that landline or WiFi internet connection will not be available.

Such alert will be sent to the horse owner and its veterinarian.

Less urgent conditions such as, slight suspected lameness, will be sent in a less urgent means.

Messages related to trimming issues will be sent to the owner and to the hoof trimmer, on a routine non-urgent basis.

Alerts related to the performance of the horse such as, under-speeding, slow response, statistically significant deviation of performance relative to the horse baseline, such alerts will be transmitted to the horse owner and to its trainer. In summary, the system will have a classification of the pertinent detected issue with a prioritized alert generation algorithm. The owner will be able to user-configure the variety of alerts, their urgency and priority as well as to whom they will be sent.

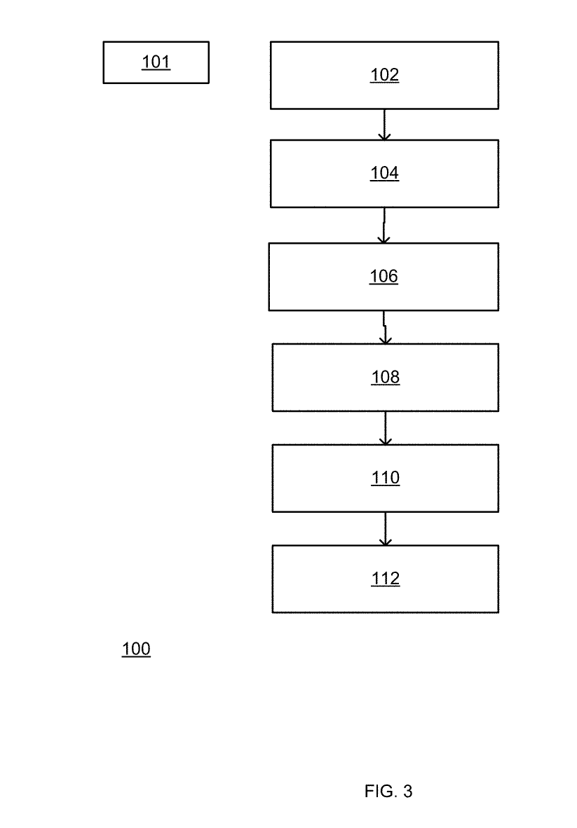

FIG. 3 illustrates method 100 for evaluating a motion related parameter of a horse.

Method 100 may include at least some of the following steps—101, 102, 104, 106, 108, 110 and 112.

Step 101 may include training a machine learning process by on a training set that may include (a) sensor information that represent a desired motion related parameter and (b) sensor information that represents deviations from the desired motion related parameter.

Step 101 may include performing supervised learning or unsupervised learning.

Step 102 may include generating, by multiple sensing units attached to multiple legs of a horse, sensor information.

The sensor information may include at least one out of motion related information and additional information.

Additionally or alternatively, the additional information may be provided from a source that differs than the multiple sensing units attached to the multiple legs of the horse.

The additional information may include at least one out of:
a. Any physiological information such as temperature, blood pressure, heart rate, and the like.
b. Terrain information. This describes the terrain (slope, rigidness) on which the horse propagates.
c. Feedback received from a third party and relates to a health or performance of the horse.
d. Ambient condition information.
e. Visual information.

Step 104 may include encrypting the sensor information to generate encrypted sensor information. This can be done by sensing units 20 and/or by communication unit 30.

Step 106 may include transmitting the encrypted information. This can be done by communication unit 30.

Step 108 may include receiving, by a remote computer, the encrypted sensor information. The remote computer may be the cloud computerized system 40

Step 110 may include decrypting the encrypted sensor information to provide decrypted sensor information.

Step 112 may include evaluating, by the remote computer, the motion related parameter of the horse by applying a machine learning process trained on a training set that may include (a) sensor information that represent a desired motion related parameter and (b) sensor information that represents deviations from the desired motion related parameter.

The motion related parameter of the horse may be a health of the horse, a state of one or more hoofs of the horse, a measure of a limping of the horse, any parameter that directly or indirectly describes the motion of the horse or any parameter that may be reflected by the motion of the horse.

Step 112 may be followed by scheduling a hoof trimming process based on the state of one or more hoofs of the horse.

Method 100 may also include deleting the sensor information shortly (for example few seconds, few minutes) after the transmitting of the encrypted sensor information or even shortly after generating the encrypted sensor information.

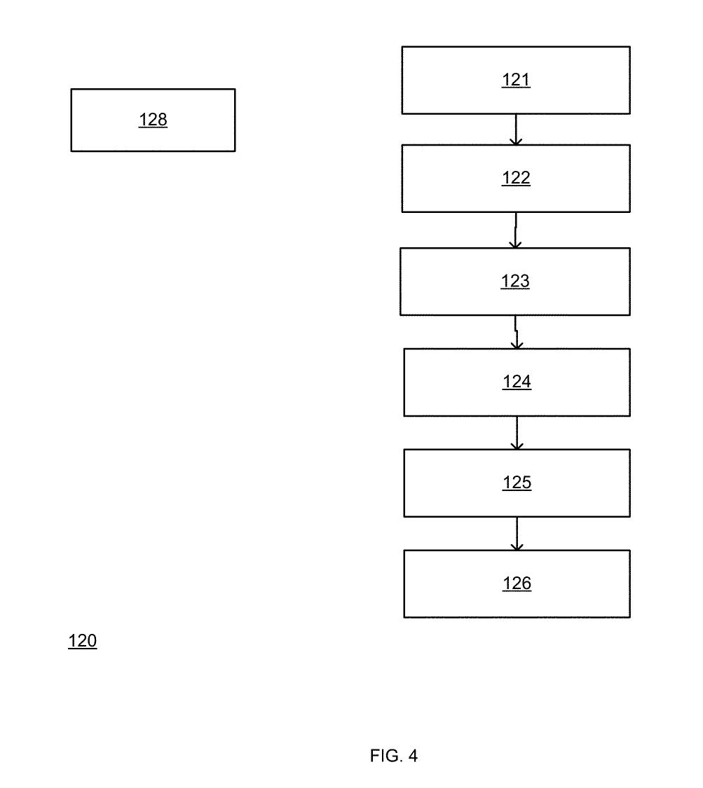

FIG. 4 illustrates method 120 for evaluating a motion related parameter of a horse.

Method 120 may include at least some of the following steps—121, 122, 123, 124, 125, 126 and 128.

Step 121 may include generating, by multiple sensing units attached to multiple legs of a horse, sensor information.

Step 122 may include encrypting the sensor information to generate encrypted sensor information.

Step 123 may include transmitting the encrypted data.

Step 124 may include receiving, by a remote computer, the encrypted sensor information.

Step 125 may include decrypting the encrypted sensor information to provide decrypted sensor information.

Step 126 may include evaluating, by the remote computer, the motion related parameter of the horse by comparing sensor information related to two or more legs of the horses.

Step 126 may include at least some of the steps:
a. Comparing between a first stance period of a first leg of the horse and a second stance period of an opposite leg of the horse.
b. Determining that the horse limps if there is an above threshold difference between the first stance period and the second stance period.
c. Determining that the horse suffers from colic when some of the legs are static while another leg repetitively kicks.
d. Searching for differences between gait rhythms of different legs that are indicative of limping.

Method 120 may also include step 128 of finding sensor information that predicts an injury by: (i) searching for a feedback that was given at a certain point in time and is indicative of an injury of a certain horse, the feedback was recognized by a horse caretaker, (ii) finding sensor information associated with the certain horse and the certain point in time, (iii) searching for sensor information of the certain horse that was obtained before that certain point of time, (iv) and determining whether the sensor information of the certain horse that was obtained before that certain point of time is usable as a predictor.

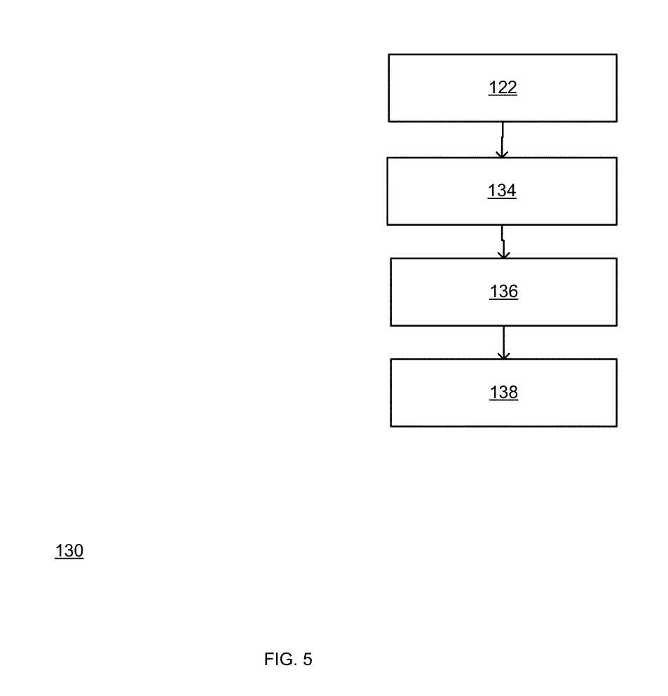

FIG. 5 illustrates method 130 for learning a three dimensional trajectory of a hoof of a horse.

Method 130 may include:
a. Step 132 of integrating gyroscope readings obtained from a gyroscope that is mechanically coupled to the hoof of the horse, to provide an absolute orientation of the gyroscope in space.
b. Step 134 of converting the absolute orientation of the gyroscope in space to an orientation of the gyroscope in relation to the earth.
c. Step 136 of compensating for the gravity of the earth to provide a gravity compensated acceleration of the gyroscope in relation to the earth; and
d. Step 138 of determining the three dimensional trajectory of the hoof of the horse by applying a double integration on the gravity compensated acceleration of the gyroscope.

Note that in these specifications we have used the terminology “limb” and “hoof” interchangeably, so it should be understood that each instance of “limb” can be construed as “hoof”.

In the foregoing specification, the invention has been described with reference to specific examples of embodiments of the invention. It will, however, be evident that various modifications and changes may be made therein without departing from the broader spirit and scope of the invention as set forth in the appended claims.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

The terms “including”, “comprising”, “having”, “consisting” and “consisting essentially of” are used in an interchangeable manner. For example—any method may include at least the steps included in the figures and/or in the specification, only the steps included in the figures and/or the specification. The same applies to the pool cleaning robot and the mobile computer.

We claim:

1. A method for evaluating a motion related parameter of a horse, the method comprises:
generating, by multiple sensing units attached to multiple legs of the horse, sensor information;
transmitting the sensor information;
receiving, by a remote computer, the sensor information; and
evaluating, by the remote computer, the motion related parameter of the horse by applying, on the sensor information, a machine learning process trained on a training set that comprises (a) sensor information that represent a desired motion related parameter and (b) sensor information that represents deviations from the desired motion related parameter.

2. The method according to claim 1 wherein the machine learning process is trained using supervised learning.

3. The method according to claim 1 wherein the machine learning process is trained using an unsupervised learning.

4. The method according to claim 1 wherein the sensor information comprises motion related information.

5. The method according to claim 1 wherein the sensor information comprises motion related information and additional information.

6. The method according to claim 5 wherein the additional information is temperature information that reflects temperature of at least one organ of the horse.

7. The method according to claim 1 wherein the motion related parameter of the horse is a health of the horse.

8. The method according to claim 1 wherein the motion related parameter of the horse is a state of one or more hoofs of the horse.

9. The method according to claim 8 comprising scheduling a hoof trimming process based on the state of one or more hoofs of the horse.

10. The method according to claim 1 wherein the motion related parameter of the horse is a measure of a limping of the horse.

11. The method according to claim 1 comprising receiving additional information; and wherein the evaluating of the motion related parameter of the horse is responsive to the additional information.

12. The method according to claim 11 wherein the additional information is terrain information.

13. The method according to claim 11 wherein the additional information is feedback received from a third party and relates to a health or performance of the horse.

14. The method according to claim 11 wherein the additional information is generated by at least one sensor that does not belong to any of the multiple sensing units attached to the multiple legs of the horse.

15. The method according to claim 11 wherein the additional information is ambient condition information.

16. The method according to claim 11 wherein the additional information is visual information.

17. The method according to claim 1 comprising deleting the sensor information shortly after the transmitting of the encrypted information.

18. The method according to claim 1 comprising encrypting the sensor information to generate encrypted sensor information; transmitting the encrypted data; receiving, by a remote computer, the encrypted sensor information; and decrypting the encrypted sensor information.

19. (canceled)

20. (canceled)

21. (canceled)

22. (canceled)

23. (canceled)

24. (canceled)

25. (canceled)

26. A non-transitory computer readable medium that stores instructions for:
generating, by multiple sensing units attached to multiple legs of a horse, sensor information;
transmitting the sensor information;
receiving, by a remote computer, the sensor information; and
evaluating, by the remote computer, a motion related parameter of the horse by applying, on the sensor information, a machine learning process trained on a training set that comprises (a) sensor information that represent a desired motion related parameter and (b) sensor information that represents deviations from the desired motion related parameter.

27. The non-transitory computer readable medium according to claim 26 wherein the machine learning process is trained using supervised learning.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. A system for evaluating a motion related parameter of a horse, the system comprises: multiple sensing units attached to multiple legs of the horse, that are configured to generate sensor information; a communication unit for transmitting the sensor information; a remote computer that is configured to (i) receive the sensor information, (ii) and evaluate the motion related parameter of the horse by applying, on the sensor information, a machine learning process trained on a training set that comprises (a) sensor information that represent a desired motion related parameter and (b) sensor information that represents deviations from the desired motion related parameter.

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)
